(19) United States
(12) Patent Application Publication
(10) Pub. No.: US 2003/0207830 A1
(43) Pub. Date: Nov. 6, 2003

(54) Mutant NDP kinases for antiviral nucleotide analog activation and therapeutic uses thereof

(76) Inventors: Dominique Deville-Bonne, Paris (FR); Sarah Gallois-Montbrun, Vaucresson (FR); Veronique Giacomoni-Fernandes, Sainte-Genevieve-Des-Bois (FR); Benoit Schneider, Villejuif (FR); Michel Veron, Paris (FR)

Correspondence Address: FINNEGAN, HENDERSON, FARABOW,, GARRETT & DUNNER, L.L.P., 1300 I Street, NW, Washington, DC, 20005-3315, US

(21) Appl. No.: 10/133,628

(22) Filed: Apr. 29, 2002

(51) Int. Cl. A61K 48/00; C07H 21/04; C12P 21/02; C12N 5/06; C12N 9/12

(52) U.S. Cl. 514/44; 435/69.1; 435/194; 435/320.1; 435/325; 536/23.2

(57) ABSTRACT

A polypeptide having a nucleoside or nucleotide kinase activity, which comprises a wild-type nucleoside or nucleotide kinase mutated at at least one amino acid position within the active site of nucleoside or nucleotide kinase to increase kinase catalytic activity towards a given nucleotide or nucleoside analog compared to the wild-type nucleoside or nucleotide kinase. A polynucleotide coding for said polypeptide. Methods, including therapeutic ones, using said polypeptide and polynucleotide.

### BACKGROUND OF THE INVENTION

The invention relates to new genes encoding mutant nucleoside or nucleotide kinases and to the polypeptides encoded by these genes. The invention covers, in particular, mutated nucleoside diphosphate (NDP) kinases showing an enhanced specificity to nucleotide analogs. The invention also relates to a process of production of the mutant NDP kinases. In addition, this invention relates to the use of the mutant genes and the polypeptides encoded by the mutant genes in therapy.

Nucleotide analogs, such as dideoxynucleosides ddl (Didanosine), ddC—(Zalcitabine), AZT (Zidovudine), d4T (Stavudine), are widely used in clinics for their antiviral effects, in particular in the treatment of AIDS. These nucleoside reverse transcriptase inhibitors (“NRTIs”), lacking both the 2′ and 3′ OH groups on the ribose moiety, serve as chain terminators and are directed towards HIV reverse transcriptase. The emergence of resistances due to mutation in the HIV gene pol coding for reverse transcriptase impairs treatment efficacy. For a couple of years, these inhibitors have been combined with other non-nucleosidic inhibitors and antiproteases in multitherapies.

NRTIs need to be activated intracellularly by the kinases of the nucleotide salvage pathway. The first two activation steps are catalyzed by kinases specific for the nucleobase (Wang 1999, Van Rompay 2000), whereas the addition of the phosphate is catalyzed by nucleoside diphosphate (NDP) kinase, which exhibits little specificity towards both the nucleobase and the ribose moiety (Parks & Agarwal 1973). The NDP kinase catalytic reaction follows a bi-bi ping-pong mechanism involving a phosphorylated intermediate on a His residue according Scheme 1:

E+N₁TP←→E˜P+N₁DP (a)

(Scheme 1)

E˜P+N₂DP←→E+N₂TP (b)

All eukaryotic NDP kinases are hexamers of identical 17 kDa polypeptides. In humans, where eight isoforms have been reported, the major forms are NDPK-A and NDPK-B, displaying 88% identity, respectively, encoded by the genes nm23-H1 and nm23-H2, (FIG. 6). All known active NDP kinases present similar kinetic parameters (Gonin, 1999). In particular, the NDP kinase from the lower eukaryote Dictyostelium discoideum (Dd-NDPK) is very similar both for its structural properties and its kinetic parameters to human NDP kinases, and it has been used as a reliable model for many studies on eukaryotic NDP kinases (Janin 2000). This enzyme was indeed easier to crystallize and to purify than human NDP kinases.

Although NDP kinase has a very high turnover with natural nucleotides, its catalytic efficiency is decreased by 10,000 fold with AZT-DP or ddNDPs as compared to thymidine (Bourdais 1996). Using fluorescence stopped-flow experiments, it has been shown that the absence of a 3′ OH group in the ribose moiety results in a 10 fold reduced affinity for the Dictyostelium enzyme and a 500-1,000 fold drop in the phosphotransfer rate (Schneider, 1998). The poor activation of NRTI by NDP kinase results in low amounts of the triphosphate form of NRTI within infected cells. This is a major cause of incomplete suppression of viral DNA synthesis and allows selection of resistance mutations (Larder, 1992).

To overcome this limitation, new NRTIs with increased reactivity towards the enzymes of the activation pathway have been designed. The recently described borano-derivatives of AZT and d4T exemplify such an approach (Meyer et al., 2000). Alternatively, modification of the salvage pathway kinases may be considered to enhance their ability to specifically phosphorylate antiviral nucleotides. Directed evolution methods can be used to achieve proteins with specific characteristics. Herpes thymidine kinase, for example, was modified by random mutations using DNA shuffling (Christians, 1999).

Notwithstanding this scientific progress, there exists a need in the art for a mutant human NDP kinase with the capacity to phosphorylate a given analog of a nucleotide more than the natural one. Such a specificity switch in an NDP kinase would enhance the concentration of activated antiviral or anticancer drugs in the target cells, and would then allow decreasing of the therapeutic dose.

### SUMMARY OF THE INVENTION

Accordingly, this invention aids in fulfilling this need in the art. Knowledge of the catalytic properties and structure regarding the amino acid residues contributing to the active site allows one to use site-specific mutagenesis to improve the capability of NDP kinase. The catalytic mechanism of this enzyme has the particularity to be substrate-assisted with the hydroxyl in 3′ of the ribose being directly involved in phosphotransfer (Tepper 1999; Janin 2000; Schneider 2001). The nucleoside analogs widely used in antiviral and anticancer therapies are devoid of the 3′ OH of interest.

The present invention investigated the possibility of modifying the NDP kinase by providing a hydroxyl residue in the active site. This led to the discovery of polypeptides having NDP kinase activity, which comprise wild-type NDP kinase, or a fragment thereof, mutated at at least one amino acid position in such a way that a hydroxyl residue is provided in the active site. More particularly, the invention relates to the addition of a hydroxyl in the active site of a polypeptide having a nucleoside or nucleotide kinase activity. This addition significantly increases catalytic activity of the kinase because it apparently compensates for a missing 3′ hydroxyl group of the sugar moiety of the nucleotide analog. The eight reported isoforms of human NDP kinase are typical examples of human NDP kinases that can be employed as the basis for mutant NDP kinases of the invention.

A fragment of a wild-type kinase is a part of any length of said kinase, provided this fragment keeps a kinase activity. For instance, the NDP kinase activity of a wild-type kinase fragment can be evaluated by the methods of the Examples.

This invention also provides polynucleotides encoding the polypeptides of the invention. Preferred polynucleotides of the invention are SEQ ID NO: 6 to SEQ ID NO: 10.

In particular, this invention provides a purified polypeptide comprising an amino acid sequence (e.g., SEQ ID NOS: 1 to 5) encoded by a gene of the invention. The preferred polynucleotides SEQ ID NO: 6 to SEQ ID NO: 10 encoded these polypeptides.

This invention additionally provides purified polynucleotides comprising the nucleic acid sequences of the genes of the invention (e.g., SEQ ID NOS: 6 and 10), and nucleic acid molecules degenerate therefrom as a result of the genetic code.

Additionally, the invention includes a purified polynucleotide that hybridizes specifically under conditions of moderate stringency with a polynucleotide of the invention (e.g., SEQ ID NOS: 6 to 10).

In another embodiment of the invention, a recombinant DNA sequence comprising at least one nucleotide sequence enumerated above and under the control of regulatory elements that regulate the expression of the polypeptide in a host is provided.

In a particular embodiment, the polypeptide of the invention is a Dictyostelium discoideum (Dd) NDP kinase, which comprises a wild-type Dd NDP kinase mutated at amino acid position 119 by the substitution of asparagine for serine. In a preferred embodiment, the polypeptide of the invention is a human NDP kinase, especially isoform A or B, which comprises a wild-type human NDP kinase mutated by the substitution of asparagine for serine at the amino acid position corresponding to the amino acid position 119 of Dd NDP kinase, that is mutated at amino acid position 115. In a more preferred embodiment, said mutated NDP kinase is further mutated at the amino acid position 55 by the substitution of leucine for histidine. Preferred polypeptides of the invention are SEQ ID NO: 1-5. SEQ ID NO: 1-5 correspond to Dd NDPK N119 S, human NDPK-A N115S, human NDPK-A N115S-L55H, human NDPK-B N115S, human NDPK-B N115S-L55H, respectively.

The invention also includes a recombinant host cell comprising a polynucleotide sequence enumerated above or the recombinant vector defined above.

The invention also contemplates antibodies recognizing the polypeptides encoded by the polynucleotide sequences enumerated above.

By “polynucleotides” according to the invention is meant the sequences encoding polypeptides of the invention, including sequences referred to as SEQ ID NOS: 6 to 10, and the complementary sequences and/or the sequences of polynucleotides that hybridize to the sequences of the invention under conditions of moderate stringency. The moderate stringency conditions are defined as washing in 2×SSC at 55° C., and hybridization operated in 5×SSC at 55° C. for the human gene and 50° C. for the Dictyostelium gene.

The invention also contemplates therapeutic methods where a therapeutic effect is obtained, at least partly, by administering a mutated NDP kinase of the invention or a corresponding polynucleotide to a patient.

The invention also contemplates compositions, preferably pharmaceutical compositions, comprising a polypeptide or a vector of the invention.

### BRIEF DESCRIPTION OF THE DRAWINGS

This invention will be described in detail by reference to the drawings in which:

FIG. 1 depicts the scheme of the active site of human NDP kinase bound to TDP (Protein Data Base code: 1NUE.PDB).

FIG. 2 depicts the pre-steady-state kinetics of phosphotransfer between phosphorylated NDPK and NDP analogs:

(A) Kinetics of reaction of phosphorylated Dictyostelium wild type and N119S NDP kinases by 100 μM Acyclovir diphosphate (Acy-DP). The phosphorylated enzyme was prepared with a stoichiometry #1 as described (Deville-Bonne 1996). The kinases were preincubated with ATP in excess in buffer T (50 mM Tris-HCl, pH 7.5, 5 mM MgCl₂ and 75 mM KCl). The increase in fluorescence upon mixing the phospho-enzyme (1 μM, final concentration) with Acy-DP in buffer T at 20° C. was monitored with a stopped-flow. The solid lines represent the best fit of each curve to a monoexponential.

(B) Concentration dependence of the rate constant on Acy-DP concentation. The pseudo-first order rate constant for the reaction (kobs) was plotted against Acy-DP. Best-fit analysis indicates that data can be analyzed as a second order reaction with apparent constants of 670 M⁻¹s⁻¹ for the wild type NDP kinase (⊙) and 4,500 M⁻¹s⁻¹ for the N119S mutant (▪).

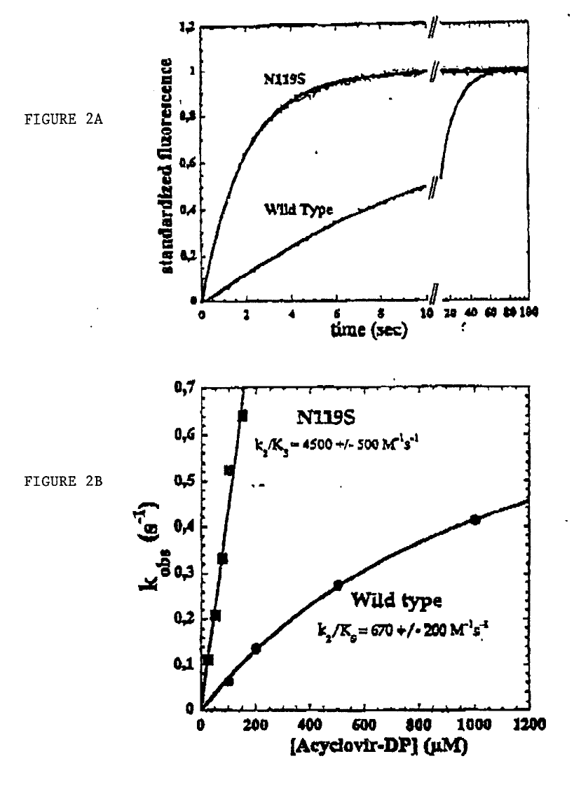

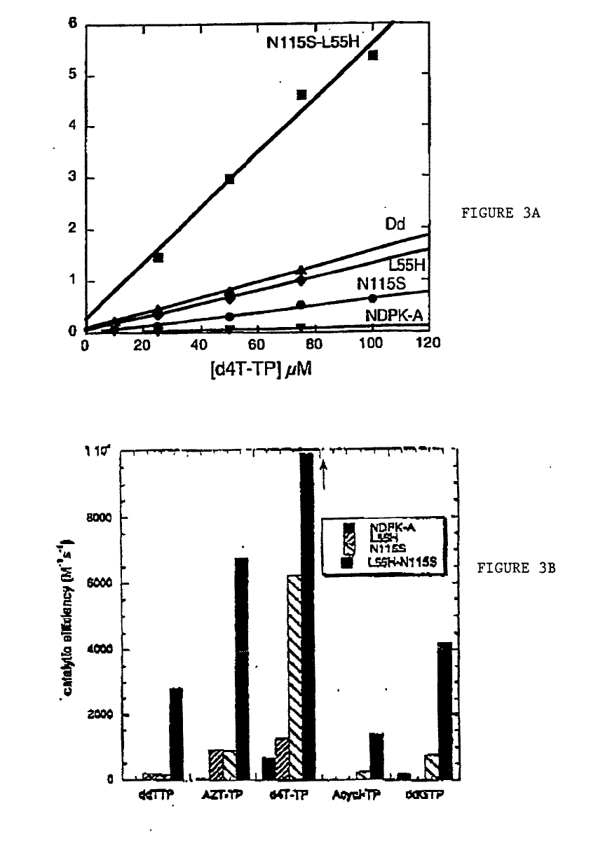

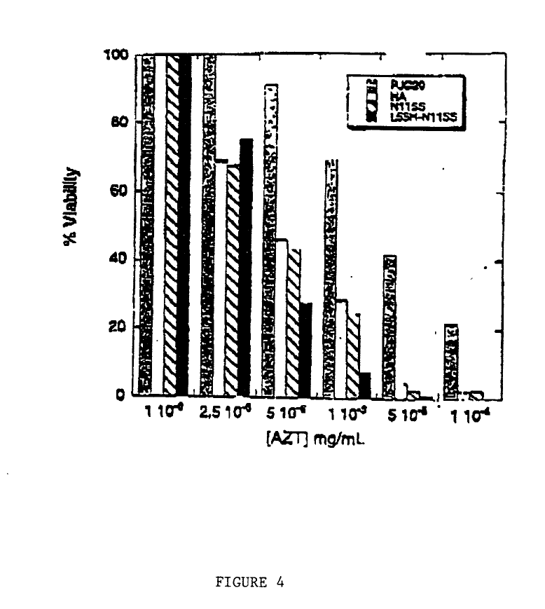

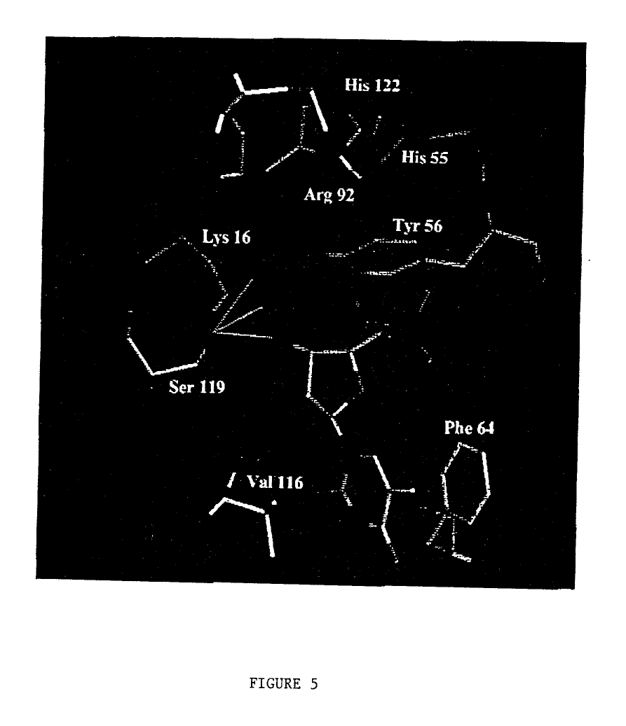

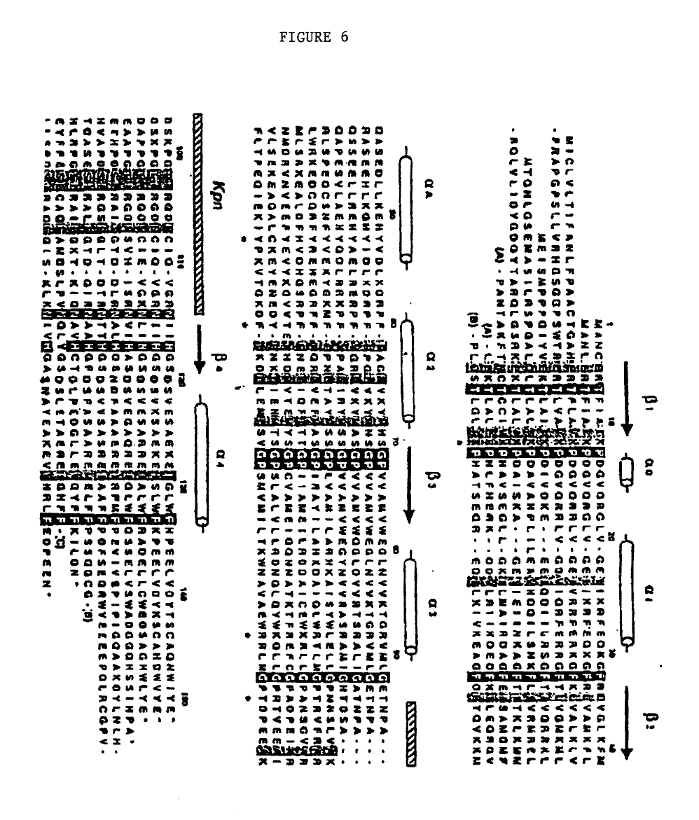

FIG. 3 depicts the catalytic efficiency of NDPK-A and mutants for nucleoside analogs. The rates of phosphorylation of pure recombinant NDP kinases (1 μM) were measured at the pre-steady state in a fluorescence stopped-flow with d4T-TP (10 to 80 μM). The catalytic efficiency (expressed in M⁻¹s⁻¹) was determined from the variation of the rate as a function of analog as shown in FIG. 2 (▾, NDPK-A, ♦ L55H, ● N115S, ▪ L55H-N115S).

FIG. 4 depicts growth inhibition by AZT of E. coli cells expressing N115S and L55H-N115S mutant human NDPK-A. E. coli (BL21 (DE3)) cells were transformed with pJC20 plasmid either empty (control) or carrying wild type NDPK-A, N115S mutant NDPK-A or L55H-N115S NDPK-A gene. The cells were grown at 37° C. in exponential phase in minimum medium. They were then complemented with various concentrations of AZT, and turbidity at 600 nm was measured after 4H. Transformation with the vector without insertion served as a control. (□): cells expressing wild type NDPK-A; ( [symbol] ): cells expressing mutant N115S-NDPK-A. (▪): cells expressing double mutant L55H-N 115S NDPK-A ( [symbol] ): cells transformed with pJC20.

FIG. 5 shows a model of the active site for the mutant NDPK-N119S of Dictyostelium with bound AZT-DP. The model was obtained starting from the published structure of the mutant NDP kinase N119A (Dd) complexed with AZT-DP (Xu et al., 1997, code 1LWX.PDB). Ala 119 was replaced by Ser and the complex was minimized using Insightll software. The Ser hydroxyl is found 3 Å far from the nitrogens of the azido group at a distance allowing H bond formation. These interactions explain the high affinity of AZT-DP to the mutant enzyme.

FIG. 6 is a primary sequence comparison between the NDP kinase domains of the human Nm23 (NDP kinase proteins).

### DETAILED DESCRIPTION OF THE INVENTION

Antiviral nucleotide analog therapies rely on the amount of the active triphosphorylated form of the analogs targeted to viral polymerase. These analogs are often slow substrates for cellular kinases of the salvage pathway, in particular for nucleoside diphosphate (NDP) kinase; the diphospho-form of antiviral analogs are phosphorylated with a 10,000 to 50,000 fold lower efficiency than natural substrates by NDP kinase. Kinetic studies with both Dictyostelium and human NDP kinases have shown that the weak catalytic efficiency is due to the absence of the sugar 3′ OH, an absolute requirement for the arrest of viral DNA chain elongation.

With the aim of improving catalytic efficiency of NDP kinases, including human NDP kinase, especially towards nucleotide analogs, mutants were engineered to provide a new hydroxyl group in the protein active site. In a preferred embodiment, the substitution of both Asn 115 for Ser and Leu 55 for His results in a human NDP kinase mutant with a 200-300 times enhanced ability to phosphorylate AZT, d4T, and acyclovir, particularly due to a higher affinity for the active site, as shown by X-ray structure. Transfection of this mutant enzyme in E. coli resulted in an increased sensitization to AZT. Such mutants are useful for gene therapies or cellular therapies.

Strategy for Improving the Enzyme Specificity Towards Nucleoside Analogs

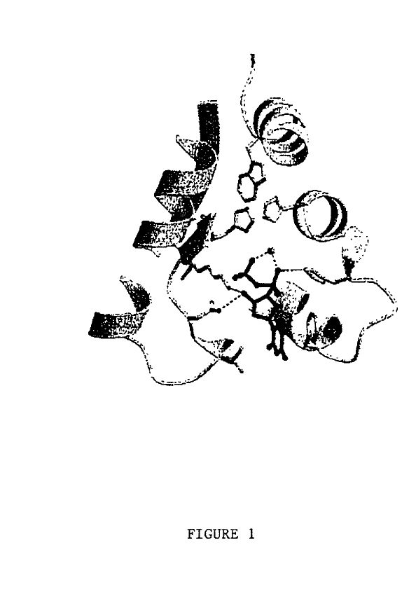

The rationale of the invention was to introduce an OH group in the NDP kinase active site at the location where it could substitute for the missing 3′ hydroxyl in nucleotide analogs. The choice of site for the introduction of an OH group in the NDP kinase active site arises from structural and catalytic considerations. The 3′ OH of the nucleotide sugar receives hydrogen bonds from two conserved protein residues, Lys 16 and Asn 119, and donates one hydrogen bond to the O₇ oxygen of the phosphate (FIG. 1). This H-bond is crucial for the catalytic efficiency of the enzyme, and its removal, in most nucleotide analogs, drastically affects catalysis (Bourdais 1996, Schneider 1998). The addition of an OH at a potential site was intended to restore, at least partially, the H-bond network between the nucleotide analog and the protein. In previous studies made with Dd-NDPK both residues Lys 16 and Asn 119 had been mutated into Ala (Schneider 2001). While the mutation N119A did not affect significantly the kinetic parameters of the enzyme, the catalytic constant of phosphotransfer by the mutated K16A was decreased by a factor of 100. Asn 119 was, therefore, a better target for mutation than Lys 16.

First, by site-directed mutagenesis, a limited set of amino acid substitutions (Ser, Thr, or Tyr) at the position of Asn 119 were introduced into the active site of Dd-NDPK. The three mutant proteins were expressed and purified to homogeneity, except N119Y, which was found unstable and poorly active (0.05% of wild type activity). The mutated NDP kinases N119T and N119S were found to phosphorylate natural substrates, such as dTDP, with a catalytic constant k_{cat}, respectively, three and ten times lower than the wild type enzyme, with little effect on K_{M} (steady state). The Ser mutation demonstrated an improvement in the enzyme reactivity for analogs, whereas the Thr mutation was without effect.

FIG. 2A shows the kinetics of phosphorylation of acyclovir diphosphate (Acy-DP) by N119S NDP kinase monitored by intrinsic fluorescence quenching (Schneider 1998). Acyclovir is an acyclic nucleoside analog of Gua used against Herpes Simplex Virus. In the presence of identical concentrations of Acy-DP, N119S NDP kinase phosphorylates Acy-DP seven times faster than the wild type enzyme. The catalytic efficiency of phosphorylation derived from the [Acy-DP] dependency of the kobs is also improved seven fold by the mutation (FIG. 2B).

Table I shows the catalytic efficiencies of phosphorylation (k₂/K_{S}) of several nucleotide analogs by the N119S mutant and the wild type Dd NDPK, as well as the affinities of the analog triphosphates for the active site bearing either Asn 119 or Ser 119.

TABLE I
Catalytic Efficiencies Of Phosphotransfer (K₂/Kₛ) And Affinities
(K_{d}) Of Dictyostelium NDP Kinase

| | k2/Kₛ | | | |
|---|---|---|---|---|
| | (M⁻¹s⁻¹) | k2/Kₛ (M⁻¹s⁻¹) | K_{D} (μM) | K_{D} (μM) |
| | wt | N119S | Inactive | Inactive-N119S |
| ATP | 4.5 × 10⁶ | 2.5 × 10⁵ | 0.2 | 2.4 |
| ddATP | 1300 | 2700 | 4.6 | 2.6 |
| GTP | 8 × 10⁶ | 7 × 10⁵ | 0.15 | 1.10 |
| ddGTP | 2300 | 3500 | 3.5 | 2 |
| acyclovirTP | 350 | 1650 | 190 | 20 |
| dTTP | 5.7 × 10⁶ | 4.3 × 10⁵ | 1.2 | 5.2 |
| AZT-TP | 270 | 1100 | 30 | 2.2 |

All numbers in italics have already been published (Schneider, 1998, 2000) The kinetic constants were measured at the pre-steady-state level using a fluorescence stopped-flow device. The binding constants were determined at equilibrium by recording the increase in fluorescence (Schneider, 2000). Values are the average of three independent determinations.

In contrast to what is observed with natural nucleotides, the Ser mutation specifically improves the catalytic efficiency with nucleotides analogs as shown in Table I; ddGTPs and AZT-TP react, respectively, 1.5 and 4 times faster with the N119S enzyme. Acy-TP is the best analog substrate of the N119S NDP kinase. Analog binding affinity was measured using a variant of Dd-NDPK (EI) as described (Schneider, 2000). This mutant (H122G-F64W) lacks the catalytic histidine, and a tryptophan replaces the phenylalanine stacking on the base in the active site (Schneider, 2000). The mutation N119S was inserted in EI in order to measure the nucleotide affinity by fluorescence titration. The Ser contribution to the binding was evaluated from the relative affinity of NTP for both enzymes (Table I). The Ser 19 reinforces the affinity for most antiviral nucleotides by a factor of 2, 10, or 15 fold, respectively, for ddGTP, Acy-TP, and AZT-TP.

Improvement of Human NDP Kinase A

The change of Asn 115 into Ser in human enzyme NDP kinase A (“NDPK-A”) was achieved using this strategy. It can be noted that, despite the similarity between Dd-NDPK and human NDPK, the Dictyostelium enzyme demonstrates higher specific activity (2000 U/mg) than the human A and B (1200-1400 U/mg) forms. This slight difference may be due to the only active site residue that is not identical; Leu-55 is replaced by His in the Dd enzyme. In order to improve the activity of the mutant human kinase, Leu 55 was replaced by His, and also, the double mutant L55H-N115S NDPK-A was engineered.

The three mutated N15S, L55H, and L55H-N115S human NDPK-A enzymes can be expressed in E. coli and purified to homogeneity. Their ability to phosphorylate natural nucleotide were studied at the steady state with ATP and dTDP. Their specific activities under standard conditions were 1900, 140, and 240 U/mg for L55H, N115S, and L55HN115S, respectively. This 1.6 fold improvement in NDPK-A activity demonstrates that the His in the 55 position is indeed responsible for the higher activity of Dd-NDPK. The replacement of Asn 115 by Ser in NDPK-A causes the same decrease in activity observed with the Dd enzyme (1/10). Replacing Asn 115 with Ser produces the same results. The double mutant activity is intermediate; the presence of His 55 improves the activity in NDPK-A and in N115S-NDPK-A by a factor of 1.7.

The intrinsic fluorescence properties of the three mutants were not affected by the mutations and were quenched upon phosphorylation of the catalytic His, as observed with wild type NDP kinase (Deville-Bonne, 1996). The quenching was somewhat lower (5%) for the enzymes carrying the mutation L55H but sufficient to monitor the phosphorylation rate of the enzymes at the pre-steady state as previously described (Schneider, 1998). All kinetics data could be fitted to monoexponentials. The phosphorylation rates kobS of N115S, L55H, and L55H-N115S mutants with the analog d4T-triphosphate were compared to the reaction with Dd-NDPK and human NDPK-A i FIG. 3A (see also FIG. 3B and Table II). The NDPK-A rate with d4T-TP is improved by factors of 1.8, 9, and 80 by mutations N115S, L55H, and the double change, respectively, while the rates for the natural nucleotide dTTP were modified by a factor of 0.08, 1.8 and 0.33. Note that the mutant L55H reacts with d4T-TP at the same rate as Dd-NDPK.

In FIG. 3B and Table II there are collected the catalytic efficiencies of several analogs of Thy (dideoxy-TTP, AZT-TP and dideoxy, didehydro-TTP) and of Guo (dideoxy-GTP and acyclovir-TP).

TABLE II
Catalytic Efficiencies Of Phosphotransfer (K₂/Kₛ) In M⁻¹s⁻¹ Of Human
NDPK-A And Mutants For Several Nucleotide Analogs Compared To
Natural Nucleotides
Specificity Change (R)

| | | | | AZT-TP | | d4T-TP | | dGTP | AcyTP | | ddGTP | |
|---|---|---|---|---|---|---|---|---|---|---|---|---|
| NDPK-A | 1.2 × 10⁶ | 20 | | 75 | | 700 | | 3.6 × 10⁶ | 25 | | 190 | |
| L55H | 2 × 10⁶ | 200 | (6) | 930 | (8) | 1280 | (10) | 6.8 × 10⁶ | 160 | (3.4) | | |
| N115S | 1 × 10⁵ | 170 | (100) | 900 | (125) | 6250 | (120) | 2.4 × 10⁵ | 250 | (150) | 750 | (60) |
| L55H-N115S | 4.5 × 10⁵ | 2800 | (370) | 6770 | (240) | 55400 | (210) | 5 × 10⁵ | 2600 | (460) | 4200 | (160) |

The activity assays were performed at the pre-steady state level.

R, the specificity change, is the ratio of the catalytic efficiencies (for analog versus natural nucleotide) of the mutant compared to the wild type enzyme.

At first glance, it is clear that each single mutation causes an increment in activity between 2 and 10 fold, and that the double mutant demonstrates additive effects with improvements 80-100 fold. The best catalytic efficiency for an analog is observed for the double mutant, which reacts with d4T-TP (5.5×10⁴ M⁻¹s⁻¹) only eight fold less than with dTTP (4.5×10⁵ M⁻¹s⁻¹), while NDPK-A reacts with d4T-TP 1700 times more slowly than with dTTP.

The mutants described so far show altered specificity for antiviral drugs and natural nucleotides. Such a switch is usually defined as R, the specificity change, reflecting the ability of a mutant enzyme to prefer the analog rather than the natural nucleotide according to the expression:

$$R = \frac{{[ \frac{{CE}^{drug}}{{E}^{nucleotide}} ]}_{mutant enzyme}}{{[ \frac{{CE}^{drug}}{{CE}^{nucleotide}} ]}_{wt enzyme}}$$

where R is the ratio of the specificity factors of the mutant compared to the original enzyme, with the specificity factor of an enzyme being defined as the ratio of the catalytic efficiencies (CE=k₂/K_{S}) for a nucleotide analog and the natural nucleotide.

The L55H mutation in human NDP kinase causes a modest switch in specificity ranging from 6 to 10. The N115S mutation causes larger switches around 100, resulting in values as high as 200 to 300 for the double mutant (Table II). Such a large specificity change factor observed with AZT and d4T suggest that the N115S and L55H mutations might be of particular interest for improving the cellular activation of AZT or d4T.

Gene transfer of such a potentiated mutant can improve the cytotoxicity of an analog toward the transfected cells; the potentiated NDP kinase would then act as a suicide gene. This allows the control of cell proliferation especially for tumor cells. For example, the 4-fold overexpression of the mitochondrial deoxyguanosine kinase in human pancreatic adenocarcinoma cell lines leads to an enhanced sensitivity of these cells to CdA, araG, and dFdG (Zhu, Karlsson, JBC 1998). Promising results have already been obtained by the combination of transfection with Herpes simplex thymidine kinase and ganciclovir, a guanosine analog (Balzarini 1985). However, the use of the Herpes thymidine kinase is limited by the immunogenicity of the viral protein (Brundiers 1999). In case of L55H-N115S NDP kinase, it is unlikely that an immunologic reaction would occur since the L55H and N115S mutations are buried inside the active site. The effect of introducing the mutant enzyme into bacterial cells supports the use of human L55H-N115S NDP kinase in gene therapy.

Effect of L55H and N115S Mutations on E. Coli Sensitivity to AZT

In a first attempt to determine whether if the expression of mutant NDP kinase makes E. coli more sensitive to antiviral drugs, the sensitivity of E. coli to nucleoside analogs was investigated. AZT and other analogs have been shown to be growth inhibitors when used at relatively high doses, presumably because their derivative-TP becomes incorporated into DNA during replication (Ono, 1989). This system has been used to determine if the presence of wild type or mutant NDP kinase increases the sensitivity of cells to AZT. AZT was choosen for this assay instead of d4T, which gave better results with the double mutant, because the level of d4T phosphorylation by thymidine kinase is very low and probably represents an important limiting step in the phosphorylation pathway of d4T (Munch-Petersen 1991).

Wild type and mutated NDP kinases were overexpressed in E. coli cells, and the sensitivity of exponentially growing cells to AZT was assayed after induction of NDP kinase expression by IPTG. The viability was estimated by plating and counting the cells. The levels of enzyme expression were checked by western blot and found similar. As shown in FIG. 4, bacteria transfected by the plasmid without insertion (pJC20) are less sensitive to AZT in the range tested than the bacteria expressing NDPK-A (HA) and mutants (N115S-NDPK-A and L55H-N1155-NDPK-A). At AZT concentrations between 5 and 10 ng/ml, fewer viable cells are found if the expressed enzyme is the double mutant rather than the wild type (wt) enzyme or the N115S mutant. The specificity change factor of the mutants of NDPK-A calculated from kinetics experiments (Table II) roughly correlates with its ability to sensitize E. coli to AZT, suggesting that the enhanced reactivity of the mutant NDPK-A towards AZT is indeed due to an increase of the phosphorylation of AZT-DP into AZT-TP in vivo.

The mutant N115S and L55H NDP kinases are useful in antiretroviral therapies, cancer chemotherapy, and cellular therapy. Gene transfer into potential HIV-target cells can help to improve both the efficacy and selectivity of nucleotide analogs. The antiviral effect was improved by a factor of 10 after transfection of the HSV TK gene into cells (HIV-1 infected human lymphoid cell line HuT 78 and monoblastoid cell line U-937) due to a increase in the triphosphate nucleotide analog (Guettari, 1997).

In the case of NDPK, substitution of Ser for Asn is optimum, and, combined with the mutation L55S, results in large specificity change (up to 300) for antiviral drugs. The extra hydroxyl decreases the affinity of natural substrates by a factor of 5 to 10 and increases the affinity for analogs by a factor of 2 to 15 (see the K_{D} for EI and EI-N119S in Table I). The 15 fold improvement is observed with AZT-TP binding; structural data may explain this result.

These NDPK-A supermutants allow the stabilization of analogs, especially AZT, but fail to mimic substrate-assisted catalysis. The better reactivity of d4T-TP compared to AZT-TP is probably related to formation of a C..H..O bond between C3′ and possibly the extra Ser, restoring the intra-nucleotide hydrogen bond (Meyer, 2000). Such improvements in specificity (R=200-300) were never reported previously. Lower R (20-50) values were reported for mutants of human thymidylate kinase obtained by site directed mutagenesis (Brundiers, 1999), for herpes thymidine kinase obtained by DNA family shuffling (Christians, 1999), and are more pronounced than that reported for TK mutants obtained by cassette mutagenesis (Munir et al, 1993) or random sequence mutagenesis (Black, 1996).

To improve the cell sensitivity, the coexpression of metabolically related genes, like the different kinases, can potentiate sensitivity to AZT or antiviral analogs (Encell, 1999). Moreover coexpression of mutants of these different genes is extremely useful.

It will be appreciated from the foregoing description that this invention has widespread applications. For example, the polypeptides of the invention are useful for the preparation of polyclonal or monoclonal antibodies that recognize the polypeptides (for example, SEQ ID NOS: 1 to 5) or fragments thereof. As used herein, the term “polypeptides of the invention” means a mutant NDP kinase of the invention, or a fragment thereof that expresses the increased kinase catalytic activity towards a given analog of a nucleotide as compared to the wild type NDP kinase. The monoclonal antibodies can be prepared from hybridomas according to the technique described by Kohler and Milstein in 1975. The polyclonal antibodies can be prepared by immunization of a mammal, especially a mouse or a rabbit, with a polypeptide according to the invention, which is combined with an adjuvant, and then by purifying specific antibodies contained in the serum of the immunized animal on a affinity chromatography column on which has previously been immobilized the polypeptide that has been used as the antigen.

Another example is the use of the polypeptides and/or polynucleotides of the invention in cellular therapy. More particularly, the cells expressing a polypeptide according the invention are rendered more sensitive to nucleotide analogs and can then be destroyed more easily. A method is to target cells to be destroyed selectively by inserting the NDP kinase of the invention or an expression vector of the same in said cells and then treating with a given nucleotide analog. Such a therapeutic method can be used in the treatment of cancer (Encell, 1999). Another method is of cellular therapy is to chose appropriate cells for the therapeutic effect intended, for example stem cells, and to render in vivo or in vitro such cells capable of expressing a mutated polypeptide according the invention. These cells are appropriate in that, for example, they express a particular epitope involved in the intended therapeutic effect. If transformed in vitro, these cells are administered to a patient. When these cells are no longer useful or become dangerous for the patient, they can be destroyed by administration of an appropriate nucleotide analog to the patient.

The mutant NDP kinases of the invention are useful for the synthesis of di- and triphospho derivatives of nucleotides and nucleotide analogs according enzymatic process. In particular, the enzymes will be coupled to an inert support resulting in an affinity column that retains the phosphate of ATP and will transfer it to XDP (Pulido-Cejudo, 1994).

Recombinant expression vectors containing a nucleic acid sequence encoding a mutant NDP kinase can be prepared using well known methods. The expression vectors include a mutant NDP kinase DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, mRNA ribosomal binding site, and appropriate sequences that control transcription and translation initiation and termination.

Nucleotide sequences are “operably linked” when the regulatory sequence functionally relates to the mutant NDP kinase DNA sequence. Thus, a promoter nucleotide sequence is operably linked to a mutant NDP kinase DNA sequence if the promoter nucleotide sequence controls the transcription of the mutant NDP kinase DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with NDP kinase polypeptides can be incorporated into expression vectors. For example, a DNA sequence for a signal peptide (secretory leader) can be fused in-frame to the mutant NDP kinase nucleotide sequence so that the mutant NDP kinase is initially translated as a fusion protein comprising the signal peptide. A signal peptide that is functional in the intended host cells enhances extracellular secretion of the mutant NDP kinase. The signal peptide can be cleaved from the mutant NDP kinase upon secretion of the kinase from the cell.

Suitable host cells for expression of mutant NDP kinases include prokaryotes, yeast, or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems can also be employed to produce mutant NDP kinase polypeptides using RNAs derived from DNA constructs disclosed herein.

Prokaryotes include gram-negative or gram-positive organisms, for example, E. coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a mutant NDP kinase polypeptide can include an N-terminal methionine residue to facilitate expression of the recombinant polypeptide in the prokaryotic host cell. The N-terminal Met can be cleaved from the expressed recombinant mutant NDP kinase polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an auxotrophic requirement. Examples of useful expression vectors for prokaryotic host cells include those derived from commercially available plasmids, such as the cloning vector pBR322 (ATCC 37017). pBR322 contains genes for ampicillin and tetracycline resistance and thus provides simple means for identifying transformed cells. To construct an expression vector using pBR322, an appropriate promoter and a mutant NDP kinase DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA). Still other commercially available vectors include those that are specifically designed for the expression of protein. These include pMAL-p2 and pMAL-c2 vectors that are used for the expression of proteins fused to maltose binding protein (New England Biolabs, Beverly, Mass., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982). A useful prokaryotic host cell expression system employs a phage λ PL promoter and a cI857ts thermolabile repressor sequence. Plasmid vectors available from the American Type Culture Collection, which incorporate derivatives of the λ PL promoter, include plasmid pHUB2 (resident in E. coli strain JMB9 (ATCC 37092)) and pPLc28 (resident in E. coli RR1 (ATCC 53082)).

Mutant NDP kinase DNA may be cloned in-frame into the multiple cloning site of an ordinary bacterial expression vector. Ideally the vector would contain an inducible promoter upstream of the cloning site, such that addition of an inducer leads to high-level production of the recombinant protein at a time of the investigator's choosing. For some proteins, expression levels may be boosted by incorporation of codons encoding a fusion partner (such as hexahistidine) between the promoter and the gene of interest. The resulting expression plasmid may be propagated in a variety of strains of E. coli.

Mutant NDP kinase polypeptides alternatively can be expressed in yeast host cells, preferably from the Saccharomyces genus (e.g., S. cerevisiae). Other genera of yeast, such as Pichia, K. lectis, or Kluyveromyces, can also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980), or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Hitzeman, EPA-73,657 or in Fleer et. al., Gene, 107:285-195 (1991); and van den Berg et. al., Bio/Technology, 8:135-139 (1990). Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli can be constructed by inserting DNA sequences from pBR322 for selection and replication in E. Coli (Ampr gene and origin of replication) into the above-described yeast vectors.

The yeast α-factor leader sequence can be employed to direct secretion of a mutant NDP kinase polypeptide. The α-factor leader sequence is often inserted between the promoter sequence and the structural gene sequence. See, e.g., Kurjan et al., Cell 30:933, 1982; Bitter et al., Proc. Natl. Acad. Sci. USA 81:5330, 1984; U.S. Pat. No. 4,546,082; and EP 324,274. Other leader sequences suitable for facilitating secretion of recombinant polypeptides from yeast hosts are known to those of skill in the art. A leader sequence can be modified near its 3′ end to contain one or more restriction sites. This will facilitate fusion of the leader sequence to the structural gene.

Mammalian or insect host cell culture systems can also be employed to express recombinant mutant NDP kinase polypeptides. Baculovirus systems for production of heterologous proteins in insect cells are reviewed by Luckow and Summers, Bio/Technology 6:47 (1988). Established cell lines of mammalian origin also can be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line (ATCC CRL 10478) derived from the African green monkey kidney cell line CVI (ATCC CCL 70) as described by McMahan et al. (EMBO J. 10: 2821,1991).

Transcriptional and translational control sequences for mammalian host cell expression vectors can be excised from viral genomes. Commonly used promoter sequences and enhancer sequences are derived from polyomavirus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113,1978; Kaufman, Meth. in Enzymology, 1990). Smaller or larger SV40 fragments can also be used.

An isolated and purified mutant NDP kinase polypeptide according to the invention can be produced by recombinant expression systems or purified from naturally occurring cells. Mutant NDP kinase polypeptides can be substantially purified, as indicated by a single protein band upon analysis by SDS-polyacrylamide gel electrophoresis (SDS-PAGE).

One process for producing mutant NDP kinase polypeptides comprises culturing a host cell transformed with an expression vector comprising a DNA sequence that encodes a mutant NDP kinase under conditions sufficient to promote expression of the mutant NDP kinase. Mutant NDP kinase polypeptide is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. For example, when expression systems that secrete the recombinant protein are employed, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose, or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify mutant NDP kinase polypeptides. Some or all of the foregoing purification steps, in various combinations, are well known and can be employed to provide an isolated and purified recombinant protein.

It is possible to utilize an affinity column comprising a mutant NDP kinase polypeptide-binding protein, such as a monoclonal antibody generated against mutant NDP kinase polypeptides, to affinity-purify expressed mutant NDP kinase polypeptides. Mutant NDP kinase polypeptides can be removed from an affinity column using conventional techniques, e.g., in a high salt elution buffer and then dialyzed into a lower salt buffer for use or by changing pH or other components depending on the affinity matrix utilized.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

The host cells of the invention can also be used in processes, for example, for testing the capacity of polypeptides of the invention to improve the metabolism of nucleoside or nucleotide analogs or for anti-viral activity.

The gene encoding the mutant NDP kinase of the invention can be incorporated in a viral vector for use in gene therapy, where the expressed mutant NDP kinase produces a therapeutic effect in vivo, or in gene transfer in vivo or in vitro. Preferred viruses for gene therapy are RNA viruses, such as retroviruses and lentiviruses, and DNA viruses, such as adeno-associated virus, herpes simplex virus type 1, and adenovirus. Viruses suitable for use in gene transfer include feline immune deficiency virus, Semliki Forest virus, influenza virus, and baculovirus.

The ability of retroviral vectors to insert into the genome of mammalian cells makes the gene encoding a mutant NDP kinase particularly useful for use in the genetic therapy of genetic diseases in humans and animals. Genetic therapy typically involves (1) adding the gene encoding a mutant NDP kinase to patient cell in vivo, or (2) removing patient cells from the body, adding the gene encoding the NDP kinase to the cells, and reintroducing the cells into the body, i.e., in vitro or ex vivo gene therapy, and generally (3) administering a given nucleotide analog (prodrug) to the patient. Discussions of how to perform gene therapy in variety of cells using retroviral vectors can be found, for example, in U.S. Pat. Nos. 4,868,116, and 4,980,286, WO89/07136, published Aug. 10, 1989, EP 378,576, published Jul. 25, 1990, WO89/0534, published Jun. 15, 1989 and WO90/06997, published Jun. 28, 1990, the disclosures of which are incorporated herein by reference.

In a preferred embodiment, the present invention is also directed to vectors, for example, retroviral vectors, containing the gene encoding a mutant NDP kinase of the invention capable of being used in somatic gene therapy. These vectors include an insertion site for the gene encoding the mutant NDP kinase and are capable of expressing controlled levels of the protein derived from the gene in a wide variety of transfected cell types. One class of retroviral vectors of the invention lacks a selectable marker, thus rendering them suitable for human somatic therapy in the treatment of a variety of disease states without the co-production of marker gene products.

Vectors, such as retroviral vectors, and their uses are described in many publications, including Mann, et al., Cell 33:153-159 (1983) and Cone Mulligan, Proc. Natl. Acad. Sci. USA 81:6349-6353 (1984). Retroviral vectors can be produced by genetically manipulating retroviruses. The wild type retroviral genome and the proviral DNA have three Psi genes: the gag, the pol, and the env genes, which are flanked by two long terminal repeat (LTR) sequences. The gag gene encodes the internal structural (nucleocapsid) proteins; the po! gene encodes the RNA directed DNA polymerase (reverse transcriptase); and the env gene encodes viral envelope glycoproteins. The 5′ LTR are sequences necessary for reverse transcription of the genome (the tRNA primer binding site) and for efficient encapsidation of viral RNA into particles (the Psi site). Mulligan, R. C., In: Experimental Manipulation of Gene Expression, M. Inouye (ed), 155-173 (1983); Mann, R., et al., Cell, 33:153-159 (1983); Cone, R. D. and R. C. Mulligan, Proc. Natl. Acad. Sci. USA 81:6349-6353(1984).

If the sequences necessary for encapsidation (or packaging of retroviral RNA into infectious virions) are missing from the viral genome, the result is a cis acting defect, which prevents encapsidation of genomic RNA. However, the resulting mutant is still capable of directing the synthesis of all virion proteins. Retroviral genomes have been described from which these Psi sequences have been deleted, as well as cell lines containing the mutant genome stably integrated into the chromosome. Mulligan, R. C., In Experimental Manipulation of Gene Expression, M. Inouye (ed), 155-173 (1983); Mann, R., et al., Cell, 33:153-159 (1983); Cone, R. D. and R. C. Mulligan, Proc. Natl. Acad. Sci. USA 81:6349-6353 (1984). Additional details on available retrovirus vectors and their uses can be found in patents and patent publications including European Patent Application EPA 0 178 220, U.S. Pat. No. 4,405,712, Gilboa, Biotechniques 4:504-512 (1986) (which describes the N₂ retroviral vector). The teachings of these patents and publications are incorporated herein by reference. The vectors of the invention are especially suited for use with packaging cell lines.

Vectors, such as retroviral vectors, are particularly useful for modifying mammalian cells with the mutant NDP kinases of the invention and the genes encoding them because of the high efficiency with which the retroviral vectors “infect” target cells and integrate into the target cell genome. Additionally, retroviral vectors are highly useful because the vectors may be based on retroviruses that are capable of infecting mammalian cells from a wide variety of species and tissues.

In both in vivo and in vitro gene therapy it may be undesirable to produce the gene product of the marker gene in cells undergoing human gene somatic therapy. Therefore, it is desirable to use retroviral vectors that integrate efficiently into the genome, express desired levels of the gene encoding the mutant NDP kinase, and produce the gene product in high titers without the co-production or expression of marker product. For this purpose, one can utilize a retroviral vector comprising in operable combination, a 5′ LTR and a 3′ LTR derived from a retrovirus of interest, and an insertion site for the gene encoding a mutant NDP kinase, and wherein at least one of the gag, env or pol genes in the vector are incomplete or defective. The vector can contain a splice donor site and a splice acceptor site, wherein the splice acceptor site is located upstream from the site where the gene encoding the mutant NDP kinase is inserted. Also, the vector can contain a gag transcriptional promoter functionally positioned such that a transcript of a nucleotide sequence inserted into the insertion site is produced, and wherein the transcript comprises the gag 5′ untranslated region. The preferred vectors of the invention are lacking a selectable marker, thus, rendering them more desirable in human somatic gene therapy because a marker gene product will not be co-produced or co-expressed.

Non-viral methods of DNA delivery can also be employed with the genes encoding the mutant NDP kinase of the invention. These non-viral methods include chemical methods, such as calcium phosphate and DEAE-dextran-mediated DNA delivery, naked DNA delivery, such as the incorporation of the mutant NDP gene into a plasmid vector, in vivo delivery of naked DNA, particle bombardment, electroporation, or the use of a delivery vehicle, such as a cationic lipid and polymers.

Gene therapy and gene transfer utilizing the mutant NDP kinases of the invention and the mutant genes encoding them can be employed in the prevention or treatment of HIV-1 on HIV-2 infection. These techniques can also be employed in the study of HIV in vitro.

This invention provides a method for inhibiting the activity of a retrovirus, such as HIV-1 or HIV-2, in vivo. The method comprises administering to a host (1) a mutant NDP kinase or gene encoding a mutant NDP kinase, which is capable of exhibiting a protective effect, a curative effect, or preventing transmission of a retrovirus and generally (2) a given nucleotide analog (prodrug). The mutant NDP kinase or gene encoding a mutant NDP kinase is administered to the host in an amount sufficient to prevent or at least inhibit infection in vivo or to prevent or at least inhibit spread of the retrovirus in vivo. These effects are achieved by administering the mutant NDP kinase or gene encoding a mutant NDP kinase to the host in an effective amount, which is preferably sufficient to induce a protective response against the retrovirus in the host.

The term “recombinant” as used herein means that a protein or polypeptide employed in the invention is derived from recombinant (e.g., microbial or mammalian) expression systems. “Microbial” refers to recombinant proteins or polypeptides made in bacterial or fungal (e.g., yeast) expression systems. As a product, “recombinant microbial” defines a protein or polypeptide produced in a microbial expression system, which is essentially free of native endogenous substances. Proteins or polypeptides expressed in most bacterial cultures, e.g., E. coli, will be free of glycan. Proteins or polypeptides expressed in yeast may have a glycosylation pattern different from that expressed in mammalian cells.

The mutant NDP kinase or the gene encoding the mutant NDP kinase of this invention can be in isolated or purified form. The terms “isolated” or “purified”, as used in the context of this specification to define the purity of protein or polypeptide compositions, means that the protein or polypeptide composition is substantially free of other proteins of natural or endogenous origin and contains less than about 1% by mass of protein contaminants residual of production processes. Such compositions, however, can contain other proteins added as stabilizers, excipients, or co-therapeutics. The polypeptide is isolated if it is detectable as a single protein band in a polyacrylamide gel by silver staining.

As used herein, the term “effective amount” means an amount that imparts protection from disease, particularly infectious disease, as evidenced by the absence of clinical indications of disease, or as evidenced by absence of, or reduction in, determinants of pathogenicity, including the absence or reduction in persistence of the infectious parasite or virus in vivo, and/or the absence of pathogenesis and clinical disease, or diminished severity thereof, as compared to individuals not treated by the method of the invention.

It will be understood that a mutant NDP kinase and gene encoding a mutant NDP kinase can be used alone or in combination, and can further be combined with other prophylactic or therapeutic substances. For example, a mutant NDP kinase or a gene encoding a mutant NDP kinase can also be combined with vaccinating agents for the corresponding disease, such as immunodominant, immunopathological, and immunoprotective epitope-based vaccines, or inactivated, attenuated, or subunit vaccines. A mutant NDP kinase or gene encoding a mutant NDP kinase is generally combined with a known NRTI in an acceptable dosage. Examples of NRTIs suitable for this purpose are identified in TABLE 1.

The present invention also relates to a composition, preferably a pharmaceutical composition, comprising (a) a polypeptide or a vector of the invention (b) optionally a nucleotide analog and (c) optionally a pharmaceutically acceptable carrier.

TABLE 1

| Trade | Chemical | | Approved | Recommended |
|---|---|---|---|---|
| Name | Name | Generic Name | Indication | Dose Range |
| Retrovir | AZT | zidovudine | HIV | Adult: 500-600 mg/day |
| | | | | Pediatric: 720 mg/m²/day |
| Hivid | ddC | zalcitabine | HIV | 1.5-2.25 mg/day |
| Videx | ddl | didanosine | HIV | 250-500 mg/day |
| Zerit | d4T | stavudine | HIV | 15-80 mg/day |
| Epivir | 3TC | lamiduvine | HIV, | HIV: Adult: 300 mg/day |
| | | | chronic | Pediatric: 8 mg/kg/day |
| | | | hepatitis B | HBV: 10-100 mg/day |
| Ziagen | synthetic | abacavir | HIV | Adult: 600 mg/day |
| | | | | Pediatric: 16 mg/kg/day |
| Combivir | 3TC + AZT | lamiduvine + zidovudine | HIV | same as each drug alone |
| Trizivir | 3TC + AZT + | zidovudine + lamiduvine + | HIV | ND |
| | | abacavir | | |

These drugs have benefit for treating several diseases. AZT, in combination with other drugs, can improve the outcome of patients with metastatic colorectal cancer. It can also induce remission in patients with adult T-cell leukemia/lymphoma.

The mutant NDP kinase and gene encoding a mutant NDP kinase is employed in the method of the invention in an amount sufficient to provide an adequate concentration of drug to prevent or at least inhibit infection of the host in vivo or to prevent or at least inhibit the spread of the parasite or virus in vivo. The amount of the mutant NDP kinase or gene encoding a mutant NDP kinase thus depends upon absorption, distribution, and clearance by the host. Of course, the effectiveness of the mutant NDP kinase or gene encoding a mutant NDP kinase is dose related. The dosage of the mutant NDP kinase or gene encoding a mutant NDP kinase should be sufficient to produce a minimal detectable effect.

The dosage of the mutant NDP kinase or gene encoding a mutant NDP kinase administered to the host can be varied over wide limits. The mutant NDP kinase or gene encoding a mutant NDP kinase can be administered in the minimum quantity, which is therapeutically effective, and the dosage can be increased as desired up the maximum dosage tolerated by the patient. The mutant NDP kinase and gene encoding a mutant NDP kinase can be administered as a relatively high amount, followed by lower maintenance dose, or the mutant NDP kinase or gene encoding a mutant NDP kinase can be administered in uniform dosages.

The dosage and the frequency of administration will vary. The amount of the mutant NDP kinase or gene encoding the mutant NDP kinase administered to a human can vary such that the amount of the mutant NDP kinase in vivo will be about 1 ng per Kg of body weight to about 1 μg per Kg of body weight at the time of initial dosing. Optimum amounts can be determined with a minimum of experimentation using conventional dose-response analytical techniques or by scaling up from studies based on animal models of disease.

The dose of the mutant NDP kinase or gene encoding a mutant NDP kinase is specified in relation to an adult of average size. Thus, it will be understood that the dosage can be adjusted by 20-25% for patients with a lighter or heavier build. Similarly, the dosage for a child can be adjusted using well known dosage calculation formulas.

This invention will be described in detail in the following Examples in which natural nucleotides and dideoxynucleosides triphosphates (ddNTP) were from Roche Molecular Biochemicals. The synthesis of the diphospho- and triphospho-derivatives of AZT, d4T, and acyclovir was as described in (Bourdais, 1996). Pyruvate kinase was purchased from Fluka and lactate dehydrogenase was from Sigma.

### EXAMPLE 1

### Expression and Purification of Wild-Type and Mutated NDP Kinases

Human NDPK-A mutants were obtained by polymerase chain reaction methods using overlap extension strategy. The oligonucleotides

5′-ATACAAGTTGGCAGGAGCATTATACATGGCAGT-3′
5′-GAACACTACGTTGACCACAAGGACCGTCCATTC-3′

and their complements were used to introduce N115S and L55H mutations, respectively, into NDPK-A. Mutations in Dd-NDPK were introduced using site-directed mutagenesis (Kunkel), with the oligonucleotides

5′-ATGTTGGTAGATCCATCATCCACGGT3′,
5′ATGTTGGTAGAACCATCATCCACGGT-3′,
and
5′-ATGTTGGTAGATACATCATCCACGGT-3′,

for N119S, N119T and N119Y mutations, respectively. Altered bases as compared to the wild type sequence are underlined in bold. Sequences were checked by automatic sequencing. The mutant EI-N119S was obtained by mutation of the previously described F64W-H122G mutated NDP kinase (here called EI) (Schneider 2000).

Wild type human NDPK-A and the mutants were expressed and purified according to Schneider et al., 2000, Mol Pharm. Recombinant wild type and mutant Dd-NDPK were obtained as described (Schneid, 1998a), except for N119Y NDP kinase which was partially purified by Q sepharose FF chromatography. Each protein was characterized by SDSIPAGE electrophoresis. The concentration of 17 kDa subunits of the enzyme was either determined by Bradford assay (Bradford, 1996) or using an absorbance coefficient of ΔA²⁸⁰=1.249 for a 1 mg/mL solution of human wild type and N115S NDPK-A, and =0.55 for wild type and mutant N119S, N119T Dd-NDP kinases, respectively.

### EXAMPLE 2

### Steady-State Kinetic Experiments

The activity of NDP kinase was measured at 20° C. with ATP and dTDP as substrates using coupled enzymes (pyruvate kinase and lactate dehydrogenase) (Lascu 1993). One unit is the amount of enzyme that catalyzes the phosphotransfer of 1 μmol/min under standard conditions: [ATP]=1 mM, [dTDP]=0.2 mM. Rate constants (k_{cat}) and Michaelis constants (Km) were determined from initial velocities for two different constant ratios of nucleotide [dTDP]/[ATP]=0.05 0.1 with [ATP] varying from 0.2 to 2 mM. k_{cat} is expressed by enzyme subunit. The ratio of the ^{apparent}k_{cat}/^{apparent}K_{M} at a given concentration of the other substrate is equal to the true value of k_{cat}/K_{M} for a ping-pong enzyme.

### EXAMPLE 3

### Stopped-Flow Kinetic Experiments

As the diphosphate form of analogs were not always available, the triphosphate analogs were used to study phosphate transfer in reaction as in Scheme I. Experiments were performed with an Hi-Tech DX2 microvolume stopped-flow reaction (Schneider, 1998) at Aexc=296 nm (for Ado derivatives) or 304 mm (for other nucleotides), 2 mm excitation slit and a 320 nm cutoff filter at the emission. After mixing NDPK (1 μM) and NTP or an analogTP (10-500 μM), the intrinsic protein fluorescence was recorded for 10-200 sec. In each experiment, 400 pairs of data were recorded, and the data from 3-4 identical experiments were averaged and fitted to a number of non-linear analytical equations using the software provided by Hi-Tech. All curves fitted to single exponentials.

### EXAMPLE 4

### Model for Analysis of the Kinetic Results

The data were analyzed using the reaction scheme:

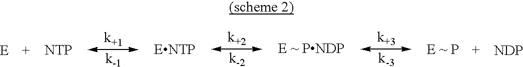

In both the forward and the reverse reactions, the product concentration remains very low, thus the product binding can be neglected, and the observed single step could be attributed to the phosphotransfer (Schneider, 1998). The rate of this observed single step is:

$${k}_{phos} = \frac{{k}_{+ 2} \cdot [ NTP ]}{( {k}_{- 1} / {k}_{+ 1} ) + [ NTP ]}$$

and reaches a limiting value (k₊₂) at NTP saturation. Saturation could not be obtained with the concentrations of any NTP used here. Therefore, catalytic efficiencies of phosphorylation (CEₚₕₒₛ=k₊₂/(k₋₁/k₊₁)=k₊₂/K_{S}) were measured, which are equivalent to second order constants and allow a reliable comparison of a variety of NDP kinase substrates.

### EXAMPLE 5

### AZT Toxicity Screening in E. Coli

The sensitivity to AZT of E. coli transformed with NDP kinase expression vectors was evaluated. Bacteriea BL21 (DE3) (Stratagene) were transformed by heat shock with pJC20 vectors (Schaertl, 1998) expressing either the wild type NDPK-A (pJC20-HA), the mutant enzyme N115S (pJC20-N115S), the double mutant enzyme L55H-N115S (pJC20-L55H-N115S), or without insertion (pJC20). Bacteria were grown at 37° C. in M9 liquid medium supplemented with casaminoacids (Dilco ref.) in exponential phase, then 10 μM IPTG was added. After 1 hour, cells were complemented with AZT from 10⁻⁷ mg/mL to 10⁻⁴ mg/mL for 4 hours. One mL of bacteria was plated onto LB agar, incubated overnight at 37° C., and counted.

In summary, this invention demonstrates that the addition of a hydroxyl group to the Asn locus at the active site of NDPK where there are several hydrogen bonds between substrate and enzyme results in a mutant with a switch in specificity in favor of antiviral analogs. One effective mutation is the replacement of Asn with Ser. It would have been expected that Tyr would have been a more effective mutation than Ser, because a Tyr residue is found in Herpes thymidine kinase (Brown, 1995,) and in T7 DNA polymerase (Doublie, 1998), in both cases near the 3′ of the sugar moiety. Moreover the mutagenesis of a Phe into a Tyr in Taq polymerase active site or in the Klenow fragment has been shown to induce a specificity change in favor of ddNTP (Tabor & Richardson 1995, Astatke, 1998). However, the N119Y mutation was, in practice, unstable and poorly active.

Plasmids containing the polynucleotides encoding the mutant NDP kinases of the invention have been deposited at the Collection Nationale de Cultures de Microorganismes (“C.N.C.M.”), 28, rue du Docteur Roux, 75724 Paris Cedex 15, France, as follows:

| Plasmid | Accession No. | Deposit Date |
|---|---|---|
| p.ndkDd-N119S | CNCM I-2850 | |
| (E. coli XL1-blue) | | |
| p.nm23H2-ndpkB-N115S | CNCM I-2851 | |
| (E. coli BL21) | | |
| BL21-NDPK A-L55H/N115S | CNCM I-2852 | |

### REFERENCES

The following publications are cited herein. The entire disclosure of each publication is relied upon and incorporated by reference herein.

Astatke, M., N. D. Grindley, et al. (1998). “How E. Coli DNA polymerase I (Klenow fragment) distinguishes between deoxy- and dideoxynucleotides.” Jounal of Molecular Biology 278(1): 147-165.

Balzarini J., E. De Clercq, et al. (1985). “Murine mammary FM3A carcinoma cells transformed with the herpes simplex virus tye 1 thymidine kinase gene am highly sensitive to the growth-inhibitory properties of (E)-5-(2-bromovinlyl-2′-deoxyuridine and related compounds.” FEBS Letters 185:95-100.

Black, M. E., T. G. Newcomb, et al. (1996). “Creation of drug-specific herpes simplex virus type 1 thymidine kinase mutants for gene therapy.” Proc, Nad. Acad. Sci, USA 93: 325-329.

Bourdais, J., R. Biondi, et al. (1996). “Cellular phosphorylation of anti-HIV nucleosides: role of nucleoside diphosphate kinase.” Journal of Biological Chemistry 271: 7887-7890.

Brown, D. G., R. Visse, et al. (1995). “Crystal structures of the thymidine kinase from herpes simplex virus type-I in complex with deoxythymidine and Ganciclovir.” Nature Structural Biology 2: 876-881.

Brundiers, R., A. Lavie, et al. (1999). “Modifying human thmidylate kinase to potentiate azidothymidine activation.” Journal of Biological Chemistry 274: 35289-35292.

Christians, F. C., L. Scapozza, et al. (1999). “Directed evolution of thymidine kinase for AZT phosphorylation using DNA family shuffling.” Natu d Biotechnology 17: 259-264.

Deville-Bonne, D., O. Sellam, et al. (1996). “Phosphorylation, of nucleoside diphosphate kinase at the active site studied by steady-state and time-resolved fluorescence.” Biochemistry 35(46): 14643-14650.

Doublie, S., S. Tabor, et al. (1998). “Crystal structure of a bacteriophage T7 DNA replication complex at 2.2 A resolution (see comments].” Nature 391(6664): 251-8.

Encell, L. P., D. M. Landis, et al. (1999). “Improving enzymes for cancer gene therapy.” Nature Biotechnolggy 17: 143-147.

Gonin, P., Y. Xu, et al. (1999). “Catalytic mechanism of nucleoside diphosphate kinase investigated using nucleotide analogues, viscosity effects and X-ray crystallography.” Biochemistry 22: 7265-7272.

Guettari, N. L. Loubiere, et al. (1997). “Use of herpes simplex Virus thymidine kinase to improve the antiviral activity of zidovudine.” Virology 235(2): 398-405.

Janin, J., C. Dumas, et al. (2000). “Three-Dimensional Structure of Nucleoside Diphosphate Kinase.” Journal of Bioenergy and Biomembane 32(3):215-225.

Larder, B. (1992). Reverse Transcriptase Inhibitors and Drug Resistance, CSHL Press.

Lascu, L, D. Deville-Bonne, et al. (1993). “Equilibrium dissociation and unfolding of nucleoside diphosphate kinase from Dictyostelium discoideum. Role of proline 100 in the stability of the hexameric enzyme.” J. Biol. Chem, 268: 20268-20275.

Meyer, P., B. Schneider, et al. 2000). “Structural basis for activation of α-boranophosphate nuclootide analogues targeting drug-resistant reverse transcriptase.” EMBO Journal 19(14): 3520-3529.

Munch-Petersen, B., L. Cloos, et al. (1991). “Diverging, substrate specificity of pure human thymidine kinases 1 and 2 against antiviral dideoxynucleosides.” J. Biol. Chem. 266: 9032-9038.

Munir, K M., D. C. French, et al. (1993). “Thymidine kinase mutants obtained by random sequence selection.” Proc. Nad. Acad. Sci. USA 90; 4012-4016.

Ono, K., H. Nagase, et al. (1989). “Differential inhibitory effects of several pyrimidine 2′, 3′-dideoxynucleoside 5′-triphosphates on the activity of reverse transcriptases and various cellular DNA polymerases.” Molecular Pharmacology 35: 578-583.

Parks, R. E. J. and R. P. Agarwal (1973). “Nucleoside diphosphokinases.” The Enzmes 8: 307-334.

Schneider, B., M. Babolat, et al. (2001). “Mechanism of phosphoryl transfer by nucleoside diphosphate kinase pH dependence and role of the active site Lys16 and Tyr56 residues.” Eur J Biochem 268(7): 1964-71.

Schneider, B., R. Biondi, et al. (2000). “The Mechanism of Phosphorylation of Anti-HIV D4T by Nucleoside Dipshosphate Kinase.” Molecular Pharmacology 57: 948-953.

Schneider, B., Y. W. Xu, et al. (1998). “Pre-steady state of reaction of nucleoside diphosphate kinase with anti-HIV nucleotides.” J. Biol. Chem 273: 11491-11497.

Tabor, S. and C. C. Richardson (1995). “A single residue in DNA polymerases of the Escherichia coli DNA polymerase I family is critical for distinguishing between deoxy- and dideoxyribonucleotides.” Proc. Natl. Acad. Sci. USA 92(14): 6339-43.

Tepper, A., H. Dammann, et al. (1994). “Investigation of the active site and conformational stability of nucleoside diphosphate kinase by site-directed mutagenesis.” Journal of Biological Chemistry 269: 32175-32180.

Van Rompay, A. R., M. Johansson, et al. (2000). “Phosphorylation of nucleosides and nucleoside analogs by mammalian nucleoside monophosphate kinases.” Pharmacology and Therapeutics 87: 189-198.

Wang, J., D. Choudhury, et al. (1999). “Stereoisomeric selectivity of human deoxyribonucleoside kinases.” Biochemistry 38(51): 16993-9.

Xu, Y., O. Sellam, et al. (1997). “X-ray analysis of azido-thymidine diphosphate binding to nucleoside diphosphate kinase.” Proc. Natl. Acad. Sci. USA 94: 7162-7165.

Zhu, C., M. Johansson, et al. (1998). “Enhanced Cytoroxicity of Nucleoside Analogs by Overexpression of Mitochondrial Deoxyguanosine kinase in Cancer Cell Lines.” Journal of Biological Chemistry 273: 14707-14711.

Pulido-Cejudo G, Gagnon J, et al. (1994) “Measurement of nucleoside diphosphate kinase-Nm23 activity by anion-exchange high-performance liquid chromatography” J. Chromatoqr. B. Biomed. Appl. Oct. 3, 1994; 660: 37-47

Bradford, M., (1976) “A rapid and sensitive method for the quantitation of microgrammes of proteins” Anal. Biochem. 72: 248-254

Schaertl, S., Konrad, M. & Geeves, M. A. (1998) “Substrate specificity of human NDP kinase reveales by transient kinetic analysis” J. Biol. Chem. 273: 5662-5669.

Encell et al. (1999) “Improving enzymes for cancer gene therapy” Nature Biotechnology 17:143-147.

What is claimed is:

1. A polypeptide having a nucleoside or nucleotide kinase activity, which comprises a wild-type nucleoside or nucleotide kinase mutated at at least one amino acid position within the active site of nucleoside or nucleotide kinase to increase kinase catalytic activity towards a given nucleotide or nucleoside analog compared to the wild-type nucleoside or nucleotide kinase.

2. The polypeptide of claim 1, wherein the increasing of the kinase catalytic activity is obtained by providing a hydroxyl residue in the active site of the nucleoside or nucleotide kinase.

3. The polypeptide of claim 2, wherein said nucleoside or nucleotide kinase is a NDP kinase.

4. The polypeptide of claim 3, wherein said NDP kinase is a Dictyostelium discoideum NDP kinase and the hydroxyl residue is provided in the active site by substitution of asparagine for serine at amino acid position 119.

5. The polypeptide as claimed in claim 4 of SEQ ID NO: 3.

6. The polypeptide of claim 3, wherein said NDP kinase is human NDP kinase and the hydroxyl residue is provided in the active site by substitution of asparagine for serine at amino acid position 115.

7. The polypeptide as claimed in claim 6 of SEQ ID NO: 1.

8. The polypeptide as claimed in claim 6 of SEQ ID NO: 4.

9. The polypeptide of claim 6, wherein said NDP kinase further comprises substitution of leucine for histidine at amino acid position 55.

10. The polypeptide as claimed in claim 9 of SEQ ID NO: 2.

11. The polypeptide as claimed in claim 9 of SEQ ID NO: 5.

12. A purified polynucleotide that encodes a polypeptide according to claim 1 to 11.

13. The purified polynucleotide of claim 12, wherein said polynucleotide encodes a polypeptide selected from SEQ ID NOS: 1 to 5.

14. A purified polynucleotide selected from SEQ ID NOS: 6 to 10.

15. A purified polynucleotide that hybridizes to either strand of a denaturated, double-stranded DNA comprising the nucleic acid molecule of any one of claims 12 or 14 under conditions of moderate stringency.

16. The purified polynucleotide as claimed in claim 15, wherein said isolated polynucleotide is derived by in vitro mutagenesis for SEQ ID NOS: 6 to 10.

17. A purified polynucleotide degenerate from the polynucleotide of claim 12 as a result of the genetic code.

18. The purified polynucleotide of claim 17, wherein said polynucleotide is generated from the polynucleotide of SEQ ID NOS: 6 to 10 as a result of the genetic code.

19. A recombinant vector that directs the expression of a polynucleotide selected from the group consisting of the polynucleotides of claims 12 to 18.

20. A purified polypeptide encoded by a polynucleotide selected from the group consisting of the polynucleotides of claims 12 to 18.

21. Purified antibodies that bind to a polypeptide of claim 20.

22. Purified antibodies according to claim 16, wherein the antibodies are monoclonal antibodies.

23. A host cell transfected or transduced with the vector of claim 19.

24. A method for the production of a polypeptide comprising culturing a host cell of claim 23 under conditions promoting expression, and recovering the polypeptide from the host cell or the culture medium.

25. A method of preventing or inhibiting infection by a retrovirus in vivo, wherein the method comprises administering to a human in need thereof (1) a polypeptide as claimed in claim 1 or a nucleic acid molecule as claimed in claim 12, and (2) a nucleotide or nucleoside analog in amounts sufficient to induce a protective response against the retrovirus in the human.

26. The method of claim 25, wherein nucleotide analogs are selected in the group consisting AZT, ddC, ddl, d4T, and 3TC.

27. The method as claimed in claim 25, wherein the human is infected with HIV-1 or HIV-2.

28. The method as claimed in claim 28, comprising administering a nucleotide analog comprising a nucleoside reverse transcriptase inhibitor (NRTI) lacking both the 2′ and 3′ OH groups on the ribose moiety in an amount sufficient to effect chain termination of HIV reverse transcriptase in the human.

29. A method of activating an NRTI in vivo, which comprises administering to a host a polypeptide as claimed in claim 1 or a nucleic acid molecule as claimed in claim 4 in an amount sufficient to increase activity of the NRTI in the host as compared to activity of the NRTI in the host in the absence of said polypeptide or nucleic acid molecule.

30. A method for the synthesis of di and triphospho derivatives of nucleotide and nucleoside analogs comprising:
(a) providing a polypeptide according to claim 1;
(b) bringing said polypeptide into contact with said nucleotide under conditions appropriate for the adequate enzymatic process to take place; and
(c) collecting the synthesized di or triphospho derivatives of nucleotide or nucleoside analogs.

31. A therapeutic method involving the selective destruction of targeted cells of a patient, wherein said method comprises the steps of targeting the cells to be destroyed by insertion of a kinase according to claim 1 or an expression vector according to claim 19 in said cells and treating said patient with a given nucleotide analog.

32. The therapeutic method of claim 31, wherein targeted cells are cancer cells.

33. The therapeutic method of claim 31, comprising providing cells capable of a given therapetic effect, inserting a kinase according to claim 1 or an expression vector according to claim 19 in said cells, observing a therapeutic effect and treating said patient with a given nucleotide analog when said cells are no longer useful.
